(19) United States
(12) Plant Patent Application Publication
(10) Pub. No.: US 2013/0031690 P1
(43) Pub. Date: Jan. 31, 2013

(54) Phlox Plant Named 'BARSEVENTYTHREE'

(50) ;

(71) Applicant: GOSEN B.H. BARTELS, Aalsmeer (NL)

(72) Inventor: GOSEN B.H. BARTELS, Aalsmeer (NL)

(73) Assignee: BARTELS BREEDING B.V., Aalsmeer (NL)

(21) Appl. No.: 13/136,384

(22) Filed: Jul. 29, 2011

(51) Int. Cl. A01H 5/00

(52) U.S. Cl. PLT/320

(57) ABSTRACT

A new phlox plant named ‘Barseventythree’ particularly distinguished by its white flowers and plant vigor, is disclosed.

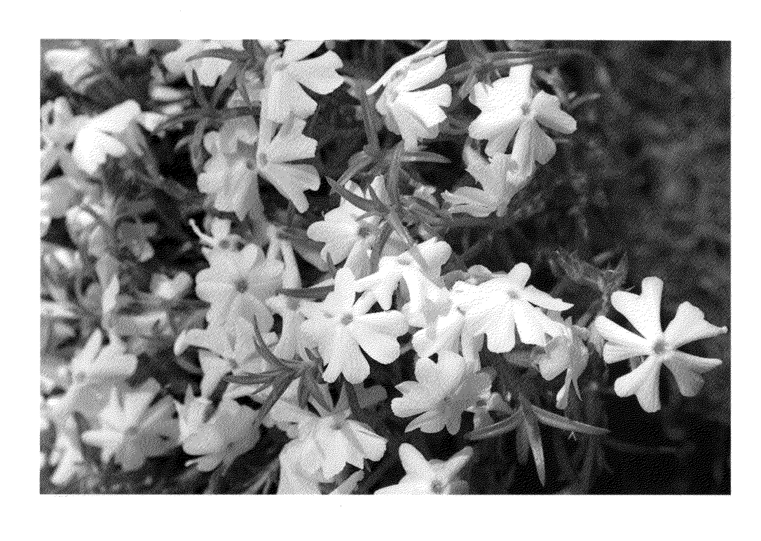

### GENUS AND SPECIES

Phlox subulata

### VARIETY DENOMINATION

‘Barseventythree’

### BACKGROUND OF THE NEW PLANT

The present invention comprises a new and distinct variety botanically known as Phlox subulata, and hereinafter referred to by the variety name ‘Barseventythree’. The new variety originated from a controlled hybridization in April of 2007 in Aalsmeer, the Netherlands between the female parent, proprietary phlox plant ‘PS024’ (unpatented), and the male parent, proprietary phlox plant ‘PS021’ (unpatented).

‘Barseventythree’ was first propagated by cuttings in Aalsmeer, The Netherlands in 2007 and has been subsequently asexually propagated. ‘Barseventythree’ has been found to retain its distinctive characteristics through successive asexual propagations via vegetative cuttings for at least 5 generations.

Plant Breeder's Rights application for ‘Barseventythree’ was filed in Europe on Jun. 23, 2011. ‘Barseventythree’ has not been sold or made publicly available more than one year prior to filing of this application.

### SUMMARY OF THE INVENTION

The following are the most outstanding and distinguishing characteristics of this new variety when grown under normal horticultural practices in Aalsmeer, The Netherlands.

1. White flowers; and

2. Abundant flowering.

### DESCRIPTION OF THE PHOTOGRAPHS

This new phlox plant is illustrated by the accompanying photograph which shows the flower blooms and foliage of the plant. The colors shown are as true as can be reasonably obtained by conventional photographic procedures. The photograph was taken in Aalsmeer, The Netherlands in February of 2011.

### DESCRIPTION OF THE NEW VARIETY

The following detailed description sets forth the distinctive characteristics of ‘Barseventythree’. The data which defines these characteristics were collected from asexual reproductions carried out in Aalsmeer, The Netherlands. The plant history was taken on 6 month old plants. Color readings were taken under natural light. Color references are primarily to the R.H.S. Colour Chart.

### DETAILED BOTANICAL DESCRIPTION

Classification:
   Family.—Polemoniaceae.
   Botanical name.—Phlox subulata.
   Common name.—Phlox.
   Denomination.—‘Barseventythree’.
Parentage:
   Female parent.—‘PS024’.
   Male parent.—‘PS021’.
Plant:
   Plant habit.—Compact, creeping, outwardly spreading and mounded.
   Growth habit.—Vigorous and freely-branching.
   Form.—Compact.
   Height (measured from the top of the soil).—10.0 cm to 20.0 cm.
   Diameter or area of spread.—20.0 cm to 30.0 cm.
   Propagation.—Vegetative cuttings.
   Time to produce a finished flowering plant.—25 to 30 weeks.
   Lastingness of inflorescence on the plant.—4 weeks.
   Outdoor plant performance.—Good.
   Time to initiate and develop roots.—3 weeks.
   Time to produce rooted cutting or liner (young plant).—5 weeks.
   Root description.—Many tiny roots.
Branches:
   Habit.—Creeping.
   Number of branches per plant.—At least 10, quickly dividing into new branches.
   Length of lateral branches.—20.0 cm to 30.0 cm.
   Diameter of lateral branches.—15.0 cm.
Stems:
   Shape in cross-section.—Round.
   Color.—RHS 199D.
   Diameter.—1.0 mm.
   Pubescence.—Absent.
Leaves:
   Arrangement.—Opposite.
   Color.—Immature leaf (both upper and lower surfaces): RHS 137C Mature leaf (both upper and lower surfaces): RHS 137A.
   Apex.—Acute.
   Base.—Truncated.
   Margin.—Smooth.
   Length.—15.0 mm.
   Width.—3.0 mm.
   Shape.—Acicular.
   Texture.—Smooth.
Peduncle:
   Color.—RHS 199D.
   Length.—25.0 mm.
   Diameter.—0.5 mm to 0.8 mm.
Inflorescence:
   Inflorescence type.—Cyme.
   Blooming habit.—Abundant flowers.
   Height.—50.0 mm.
   Width.—50.0 mm.
   Natural flowering season.—Spring (April to May).
   Flower number per inflorescence.—3 to 6.
Flowers:
   Flower Bud.—Size: 12.0 mm.
   Pedicel.—Color: RHS 137A Length: 10.0 mm Diameter: 0.5 mm.
   Sepals.—Number: 5 Shape: Fused Length: 10.0 mm Width: 2.0 mm Color (both upper and lower surfaces): RHS 137A.
   Corolla.—Length: 12.0 mm Width: 3.0 mm.
   Petals.—Length: 15.0 mm Width: 9.0 mm Color: Upper surface (base color and at center): RHS 155C Outside of corolla tube: RHS 155C.
Reproductive Organs:
   Stamens.—Quantity (per flower): 5 Anther color: RHS 12C Pollen color: RHS 12C.
   Pistil.—Quantity: 1 Color: RHS 137A Size: 2.0 mm.
   Style.—Length: 8.0 mm. Shape: Three lobes.
Disease and Insect Resistance: Not observed
Pest resistance: Not observed

### COMPARISON WITH PARENTAL AND COMMERCIAL VARIETIES

‘Barseventythree’ differs from the female parent ‘PS024’ (unpatented) in that ‘Barseventythree’ has white flowers (RHS 155C), whereas ‘PS024’ has pink flowers (RHS 69A).

‘Barseventythree’ differs from the commercial variety ‘Maischnee’ (unpatented) in that ‘Barseventythree’ has a plant height of 10 cm to 20 cm, and a flower size of 1.5 cm, while ‘Maischnee’ has a plant height of 10 cm, and a flower size of 1.0 cm. Additionally, ‘Barseventythree’ is a more vigorous and a more abundant flowering plant than ‘Maischnee’.

I claim:

1. A new and distinct variety of phlox plant named ‘Barseventythree’ as shown and described herein.
